(19) United States
(12) Patent Application Publication
(10) Pub. No.: US 2013/0052158 A1
(43) Pub. Date: Feb. 28, 2013

(54) USE OF AUTOLOGOUS EFFECTOR CELLS FOR TREATMENT OF MULTIPLE MYELOMA

(71) Applicant: Frits Van Rhee, Little Rock, AR (US)

(72) Inventor: Frits Van Rhee, Little Rock, AR (US)

(73) Assignee: University of Arkansas for Medical Science, Little Rock, AR (US)

(21) Appl. No.: 13/505,050

(22) PCT Filed: Oct. 30, 2009
(86) PCT No.: PCT/US09/62868

§ 371 Date: Nov. 14, 2012

(51) Int. Cl. A61K 35/14; A61K 38/20; A61M 5/00; A61P 35/00

(52) U.S. Cl. 424/85.2; 424/93.71; 604/522

(57) ABSTRACT

The present disclosure provides methods for treating multiple myeloma using autologous expanded and activated NK cells.

### 1. BACKGROUND

Multiple myeloma (also referred to herein as “myeloma”) is a malignant proliferation of plasma cells that produce monoclonal immunoglobulin. The myeloma tumor, its products, and the host response to it result in symptoms including persistent bone pain or fracture, renal failure, susceptibility to infection, anemia, hypercalcemia, and occasionally clotting abnormalities, neurologic symptoms and vascular manifestations of hyperviscosity. (See D. Longo, in Harrison's Principles of Internal Medicine 14th Edition 713 (McGraw-Hill, New York, 1998)). Multiple myeloma is a progressive and incurable disease that affects 14,400 new individuals in the United States annually (See Anderson et al. (1999) Introduction. Seminars in Oncology 26:1).

Multiple myeloma is difficult to diagnose early because there may be no symptoms in the early stages. Furthermore, no effective long-term treatment currently exists for the disease. The median duration of survival is six months when no treatment is given. The main treatment for multiple myeloma is systemic chemotherapy with agents such as melphalan, thalidomide, cyclophosphamide, doxorubicin, lenalidomide (Revlimid®), or bortezomib (Velcade®), either alone or in combination. However, some patients do not respond to chemotherapy. The current median of survival is greater than 5 years as a result of advances in treatment. Nevertheless, fewer than 5% of patients live longer than 10 years (See Anderson et al. (1999) Annual Meeting Report 1999. Recent Advances in the Biology and Treatment of Multiple Myeloma).

Additional treatment strategies include high-dose therapy with autologous hematopoietic cell transplantation (HCT), tandem autografts, and high-dose conditioning with allogeneic HCT. Allogeneic HCT is associated with a higher frequency of sustained remissions and a lower risk of relapse due to the graft-versus-tumor activity resulting from immune response to minor antigen differences between donor and host. Unfortunately, allogeneic HCT is also associated with high transplantation-related mortality, due in part to graft versus host disease (GVHD). Approaches using nonmyeloablative conditioning and novel posttransplantation immunosuppression to assure engraftment and graft-versus-tumor effects have reduced the transplantation related mortality. (See, e.g., Maloney, et al. (2003) Blood 102:3447).

Recently, killer immunoglobulin-like receptor-ligand mismatched natural killer (“NK”) cell transfusions from haplo-identical donors achieved near complete remission in 50% of multiple myeloma patients in the trial. (Shi et al. (2008) Brit. J. Haemotol. 143:641). Nevertheless, 2 out of the 10 patients in this study had progressive disease, and the median duration of response was only 105 days for the other 8 patients.

There is a need for additional multiple myeloma therapies that do not rely on the availability of appropriate donors, that effectively kill myeloma cells without killing normal cells, and that do not elicit early rejection in patients.

### 2. SUMMARY

Multiple myeloma is a progressive and at present incurable cancer of the plasma cells. Current therapies are aimed at the amelioration of myeloma symptoms and long term survival. A recent trial utilizing IL-2 activated, killer immunoglobulin-like receptor-ligand mismatched natural killer (“NK”) cell transfusions from haplo-identical donors yielded a near complete response in 50% of multiple myeloma patients (Shi et al. (2008) Brit. J. Haemotol. 143:641). However, 5 of the 10 patients relapsed early (31-133 days) after NK cell infusion and 2 had progressive disease, which could have been due to an insufficient dose of NK cells or early rejection. Furthermore, appropriate NK cell donors were found for only 30% of patients who were otherwise eligible for the trial.

Accordingly, described herein are methods of treating multiple myeloma by administering to a patient in need thereof a therapeutically effective amount of expanded and activated autologous NK cells, wherein the NK cells are administered in the absence of an antibody that targets NK cells and of an antibody that targets myeloma cells. An antibody that targets NK cells refers to an antibody that targets an antigen on the surface of NK cells such as the killer-cell immunoglobulin-like receptor (KIR). An antibody that targets myeloma cells refers to an antibody that targets an antigen on the myeloma cell surface, such as CD20, CD38, CD40, CD56, CD74, CD138, CD317 (also known as HM1.24 antigen), IGF receptor, IL-6 receptor or TRAIL receptor. The monotherapy described herein can be administered with other therapeutic agents, for example in combination with chemotherapeutic agents. Specific therapeutic regimens are provided herein. Patients with multiple myeloma at any stage can benefit from treatments in accordance with the methods described herein.

All publications mentioned in this specification are herein incorporated by reference. Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

The features and advantages of the disclosure will become further apparent from the following detailed description of embodiments thereof.

It should be noted that the indefinite articles “a” and “an” and the definite article “the” are used in the present application, as is common in patent applications, to mean one or more unless the context clearly dictates otherwise. Further, the term “or” is used in the present application, as is common in patent applications, to mean the disjunctive “or” or the conjunctive “and.”

### 3. BRIEF DESCRIPTION OF THE FIGURES

FIG. 1 shows the percentage of NK cells, T-cells and NKT cells present at 0, 7 and 14 days of ex vivo co-culture of PBMCs from myeloma patients with K562-mb15-41BBL cells.

FIG. 2 shows the fold-increase in the number of NK cells and T-cells from four patients with multiple myeloma after 14-days of co-culturing with K562-mb15-41BBL cells.

FIG. 3 demonstrates the level of expression of CD3 and CD56 on the surface of NK cells from four patients with multiple myeloma before and after ex vivo expansion.

FIG. 4 shows the immunophenotype of expanded NK cells from multiple myeloma patients.

FIG. 5 shows in vitro specific lysis of cells from multiple myeloma patients upon exposure to non-expanded and expanded autologous NK cells.

FIG. 6 shows the distribution of expanded NK cells from multiple myeloma patients in the bodies of NOD-SKID mice at 0, 4 and 48 hours after injection into the tail vein.

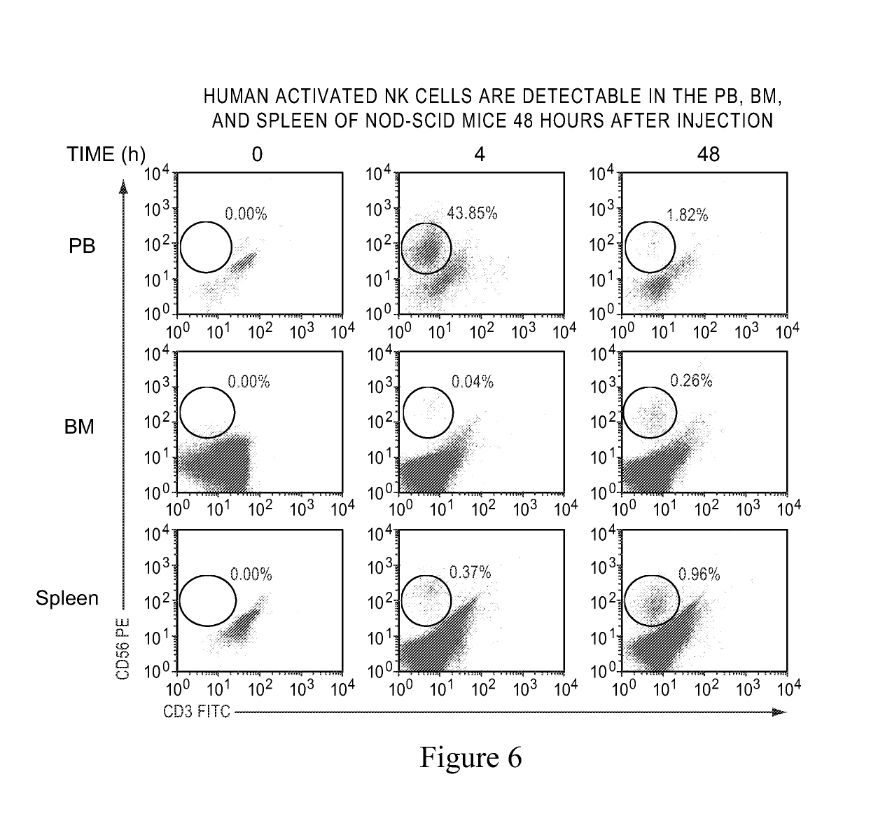

### 4. DETAILED DESCRIPTION

The present disclosure relates to compositions and methods for treating multiple myeloma in a subject. Specifically, the present disclosure relates to the treatment of multiple myeloma in a subject by administering an effective amount of autologous effector cells, in particular, autologous NK cells.

A “subject” or “patient” to whom the combination therapy is administered can be a mammal, such as a non-primate (e.g., cow, pig, horse, cat, dog, rat, etc.) or a primate (e.g., monkey or human).

Treatment of multiple myeloma includes the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

4.1 Autologous Effector Cells

The present disclosure relates to the use of expanded autologous effector cells from a subject with multiple myeloma to treat multiple myeloma. In certain aspects, the present disclosure relates to the use of expanded autologous effector cells from a subject with multiple myeloma. In certain embodiments, the effector cells for use in the methods of the disclosure are autologous lymphoid cells, i.e., lymphoid cells from the subject to be treated. In particular embodiments, the autologous lymphoid cells are natural killer (“NK”) cells.

In certain embodiments, NK cells are obtained from peripheral blood mononuclear cells (“PMBCs”) of the subject to be treated. In particular embodiments, the NK cells are expanded. The term “expanded” as used herein in the context of effector cells (i.e., NK cells) refers to effector cells that are cultured under conditions that promote (i) an increase in the total number of effector cells relative to the number in the starting culture and (ii) the activation of the effector cells. The terms “activate” or “activated” as used herein in relation to effector cells refer to inducing a change in their biologic state by which the cells express activation markers, produce cytokines, proliferate and/or become cytotoxic to target cells. Typically, NK cells are expanded and activated under the culturing conditions described herein. In particular embodiments, culturing conditions used to expand and activate NK cells in a mixed culture (e.g., PMBCs) promote activation of NK cells but not of T-cells or NKT-cells.

In certain embodiments, PBMCs are cultured under conditions that promote an increase in the fraction of NK cells and a decrease in the fraction of T-cells and/or NKT cells relative to the starting culture. In some embodiments, PBMCs are cultured under conditions that promote an increase in the fraction of NK cells in the culture and no increase or decrease in the fraction of T-cells and/or NKT cells in the culture relative to the starting culture. In particular embodiments, PBMCs are cultured under conditions that promote expansion of NK cells so that NK cells are the largest fraction of cells in the culture. In various embodiments, NK cells lacking T-cell receptors (CD56⁺ CD3⁻ cells) are preferentially expanded.

In some embodiments, NK cells are at least about 10% of the total cell population at the end of the culturing period. In various embodiments, NK cells are at least about 15% of the total cell population, such as at least about 20%, such as at least about 25%, such as at least about 30%, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 96%, such as at least about 97%, such as at least about 98%, or such as at least about 99% of the total cell population at the end of the culturing period, or a percentage of the total cell population ranging between any of the foregoing values (e.g., NK cells are from at least about 50% to at least about 70% of the total cell population at the end of the culturing period).

In particular embodiments, NK cell expansion is about 10-fold at the end of the culturing period relative to the number of NK cells in the starting cell culture. In various embodiments, NK cell expansion is at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 55-fold, such as at least about 60-fold, such as at least about 65-fold, such as at least about 70-fold, such as at least about 75-fold, such as at least about 80-fold, such as at least about 85-fold, such as at least about 90-fold, such as at least about 95-fold, such as at least about 100-fold, such as at least about 150-fold, such as at least about 200-fold, such as at least about 250-fold, such as at least about 300-fold, such as at least about 350-fold, such as at least about 400-fold, such as at least about 500-fold, such as at least about 600-fold, such as at least about 750-fold, such as at least about 1000-fold, such as at least about 5000-fold, such as at least about 7500-fold, such as at least about 10,000-fold or more at the end of the culturing period relative to the number of NK cells in the starting culture, or a fold-value ranging between any of the foregoing values (e.g., NK cell expansion is from at least about 95-fold to at least about 200-fold at the end of the culturing period).

Expansion and activation of NK cells can be accomplished by any method known in the art. (See e.g, Cho et al. (2009) Korean J. Lab. Med. 29:89 and U.S. Patent Publication No. 2006/0093605, each of which is incorporated herein by reference in its entirety). In some embodiments, NK cells, e.g., in PBMCs, are cultured in the presence of stimulatory cytokines. Such cytokines include, but are not limited to, IL-2, IL-4, IL-7, IL-12 and IL-15, either alone or in combination. In other embodiments, NK cells are expanded and activated by culturing the cells in the presence of stimulatory molecules such as an anti-CD3 antibody and IL-2.

Expansion and activation of NK cells can also be accomplished by co-culturing the cells with accessory cells. In certain embodiments, such accessory cells include, but are not limited to, monocytes, B-lymphblastoid cells, HFWT cells (a Wilms tumor-derived cell line), allogeneic mononuclear cells, autologous lymphocytes, mitogen activated lymphocytes and umbilical cord mesenchymal cells. In various embodiments, the accessory cells are K562 cells, a cell line derived from a patient with myeloid blast crisis of chronic myelogenous leukemia and bearing the BCR-ABL1 translocation. In certain embodiments, NK cells are co-cultured with accessory cells alone or in the presence of one or more cytokines. In certain embodiments, the cytokines are added to the culture medium. In other embodiments, the cytokines are expressed on the surface of the accessory cells.

In some embodiments, expansion and activation of NK cells are accomplished by co-culturing with accessory cells that have been modified to express NK stimulatory molecules on the cell surface. In certain embodiments, the stimulatory molecules include 4-1BBL (the ligand for 4-1BB, which is also known as CD137L), and membrane bound IL-15. In some embodiments, cell lines that can be modified for use as accessory cells to expand and activate NK cells include, but are not limited to, K562 cells, HFWT cells, HHUA cells (uterine endometrium cell line), HMV-II (melanoma cell line), HuH-6 (hepatoblastoma cell line), Lu-130 and Lu-134-A (small cell lung carcinoma cell lines), NB19 and NB69 (neuroblastoma cell lines), NEC14 (embryonal carcinoma cell line), TCO-2 (cervical carcinoma cell line) and TNB1 (neuroblastoma cell line). In particular embodiments, the cell line used as accessory cells in co-culture does not express or poorly expresses both MHC I and MHC II molecules. In certain embodiments, the accessory cells are K562 cells modified to express 4-1BBL and membrane-bound IL-15. In some embodiments, the accessory cell is K562-mb15-41BBL. (See Cho et al. (2009) Korean J. Lab. Med. 29:89-96, which is incorporated herein by reference in its entirety).

In some embodiments, the co-culture is started with a 1:1 ratio of accessory cells to CD56⁺CD3⁻ cells in the culture. In other embodiments, the co-culture is started with a 2:1 ratio, a 3:1 ratio, a 4:1 ratio, a 5:1 ratio, a 6:1 ratio, a 7:1 ratio, an 8:1 ratio, a 9:1 ratio, a10:1 ratio, an 11:1 ratio, a 12:1 ratio, a 13:1 ratio, a 14:1 ratio or a 15:1 ratio of accessory cells to CD56⁺CD3⁻ cells in the culture. The number of viable CD56⁺CD3⁻ cells in a culture can be quantified by any method known in the art, including, but not limited to, Trypan-blue dye exclusion and by flow cytometry using labeled antibodies for CD56. In certain embodiments, co-cultures are maintained for less than 24 hours, such as for about 4 hours, about 6 hours, about 8 hours, about 10 hours, about 12 hours, about 14 hours, about 16 hours, about 18 hours or about 20 hours. In other embodiments, co-cultures are maintained for about 1 week, for about 2 weeks or for about 3 weeks. In some embodiments, co-cultures are maintained for a period of time ranging between any two of the foregoing values (e.g., co-cultures are maintained for about 8 hours to about 18 hours). In particular embodiments, co-cultures are maintained for 2 weeks. It will be understood by the skilled artisan that prolonging the time of co-culture will increase the number of autologous NK cells. Thus, it is within the skill in the art to adjust the time of co-culture based on the desired level of expansion and activation of the NK cells. In various embodiments, in order to prevent overgrowth of accessory cells, the co-culture is irradiated at doses of, e.g., 30 Gy, 50 Gy, 70 Gy, or 100 Gy.

NK cells can be expanded using reagents and culture conditions known in the art. An exemplary protocol for obtaining clinical-grade purified functional NK cells for infusion is set forth in Cho et al. (2009) Korean J. Lab. Med. 29:89-96 and the references cited therein, which is incorporated herein by reference in its entirety.

In certain embodiments, activated NK cells are genetically modified after expansion to express artificial receptors directed against molecules that are present on the surface of cancer cells. In various embodiments, NK cells are re-stimulated after genetic modification, e.g., by co-culturing the genetically modified NK cells with accessory cells. Such genetic modification of activated NK cells can be accomplished by any method known in the art. In some embodiments, genetic modification of NK cells can be accomplished by transduction with retroviruses carrying plasmids that encode artificial receptor molecules. (See, e.g., U.S. Patent Publication No. 2006/0093605 and Imai et al. (2005) Blood 106:376-383, each of which is incorporated herein by reference in its entirety).

In some embodiments, a solid support may be used to expand and activate NK cells instead of accessory cells expressing stimulatory molecules on the cell surface. In certain embodiments, such supports will have attached on the surface one or more molecules capable of binding to NK cells and inducing activation or a proliferative response. In some embodiments, the supports are designed to bind one or more molecules that induce activation of NK cells or a proliferative response when NK cells are passed over the solid support and bind to the one or more molecules. Molecules that induce activation of or a proliferative response from NK cells include, but are not limited to CD137, IL-15, or fragments of either CD137 or IL-15 that retain the ability to induce the desired response. See U.S. Patent Publication No. 2006/0093605, which is incorporated herein by reference in its entirety.

4.2 Therapeutic Methods and Routes of Administration

Expanded and activated autologous NK cells are useful as a monotherapy for treating multiple myeloma according to the methods described herein.

Expanded autologous NK cells for use as a monotherapy are typically administered to a patient by intravenous injection or infusion. In certain embodiments, NK cells are derived from PBMCs obtained from the patient by apheresis. NK cells are expanded as described above, collected from the culture medium, washed, and suspended in a physiologically compatible carrier for injection into the patient. As used herein, the term “physiologically compatible carrier” refers to a carrier that is compatible with the other ingredients of the formulation and not deleterious to the recipient thereof. Physiologically compatible carriers are known to those of skill in the art. Examples of suitable carriers include phosphate buffered saline, Hank's balanced salt solution +/−glucose (HBSS), Ringer's solution, dextrose solution, and a solution of 5% human serum albumin in 0.9% sodium chloride for injection. In other embodiments, PBMCs are obtained from the patient, cryopreserved and thawed before NK cell expansion as described above. In some embodiments, expanded NK cells are depleted of residual T-cells by methods known in the art, e.g., using the CliniMACS System (Miltenyi) for cell selection, before administration to the patient.

In typical embodiments, an effective dose of autologous NK cells to be administered to a subject with multiple myeloma is about 1×10⁵ cells/kg of body weight, such as about 5×10⁵ cells/kg of body weight, such as about 1×10⁶ cells/kg of body weight, such as about 5×10⁶ cells/kg of body weight, such as about 1×10⁷ cells/kg of body weight, such as about 2×10⁷ cells/kg of body weight, such as about 3×10⁷ cells/kg of body weight, such as about 4×10⁷ cells/kg of body weight, such as about 5×10⁷ cells/kg of body weight, such as about 7.5×10⁷ cells/kg of body weight or such as about 1×10⁸ cells/kg of body weight. In certain embodiments an effective dose of autologous NK cells for treatment of multiple myeloma ranges between any two of the foregoing values, such as from about 1×10⁷ to about 1×10⁸ cells/kg of body weight, etc.

In certain embodiments, the dose of autologous NK cells to be administered to a subject with multiple myeloma contains less than about 1×10⁵ T-cells/kg of body weight, such as less than about 5×10⁴ T-cells/kg of body weight, such as less than about 1×10⁴ T-cells/kg of body weight, such as less than about 5×10³ T-cells/kg of body weight, such as less than about 1×10³ T-cells/kg of body weight. In certain embodiments the dose of autologous NK cells for treatment of multiple myeloma contains an amount of T-cells ranging between any two of the foregoing values, such as from less than about 1×10⁵ to less than about 1×10³ T-cells/kg of body weight, etc.

The effective dose of autologous NK cells can be administered in a single dose or in multiple doses. In certain embodiments, the effective dose of autologous NK cells is administered in a single dose by continuous intravenous administration. In certain embodiments, expanded NK cells are administered over a period of time from about 1 to about 24 hours, such as over a period of about 1 to 2 hours. Dosages can be repeated from about 1 to about 4 weeks or more, for a total of 4 or more doses. Typically, dosages are repeated once every week, once every two weeks, or once a month for a minimum of 4 doses to a maximum of 52 doses.

Determination of the effective dosage, total number of doses, and length of treatment with autologous expanded NK cells is well within the capabilities of those skilled in the art, and can be determined using a standard dose escalation study to identify the maximum tolerated dose (MTD) (see, e.g., Miller et al. (2005) Blood 105:3051; Richardson et al. (2002) Blood, 100(9):3063, the contents of which is incorporated herein by reference).

It will be recognized by one of skill in the art that the optimal quantity and spacing of individual dosages of autologous NK cells will be determined by the nature and extent of the multiple myeloma being treated, the form, route and site of administration, the age and physical condition of the particular subject being treated, and the therapeutic regimen (e.g., whether an additional therapeutic agent is used), and that the skilled artisan will readily determine the appropriate dosages and dosing schedules to be used. The dosages can be repeated as often as appropriate. If side effects develop the amount and/or frequency of the dosages can be altered or reduced, in accordance with normal clinical practice.

4.3 Combination with Other Treatment Strategies or Agents

In various embodiments, the administration of autologous NK cells is combined with another treatment strategy. In some embodiments, the monotherapy can be administered prior to the initiation of a treatment regimen incorporating stem cell transplantation. In other embodiments, the monotherapy can be administered following a treatment regimen incorporating stem cell transplantation. The stem cell transplantation regimen can be autologous or syngeneic, tandem autologous, “mini” allogeneic, and/or combinations thereof.

In still other embodiments, the autologous NK cells can be administered prior to delayed rescue with stem cells.

In some embodiments, autologous NK cells are administered before or after non-myeloablative chemotherapy with, e.g., low doses of cyclophosphamide and fludarabine or low-dose radiation.

In other embodiments, autologous NK cells are administered after conditioning therapy, such as conditioning therapy with cyclophosphamide and fludarabine or melphalan and fludarabine.

In certain embodiments, administration of autologous NK cells can precede or follow administration of an additional therapeutic agent. As a non-limiting example, the autologous NK cells and the additional therapeutic agent can be administered concurrently for a period of time, followed by a second period of time in which the administration of the autologous NK cells and the additional therapeutic agent is alternated. In certain embodiments, the additional therapeutic agent can be administered concurrently with the autologous NK cells.

Because of the potentially synergistic effects of administering either the autologous NK cells and the additional therapeutic agent, such agents can be administered in amounts that, if any of the agents is administered alone, is/are not therapeutically effective. For example, in various embodiments, the dosage of the autologous NK cells and/or the dosage of the additional therapeutic agent administered is about 10% to 90% of the generally accepted efficacious dose range for either the cells or the additional agent therapy alone. In some embodiments, about 10%, about 15%, about 25%, about 30%, about 40%, about 50%, about 60%, about 75%, or about 90% of the generally accepted efficacious dose range is used, or a dosage ranging between any of the foregoing values (e.g., 10% to 40%, 30% to 75%, or 60% to 90% of the of the generally accepted efficacious dose range) is used.

Therapeutic agents that can be used in combination with the autologous NK cells described herein include, but are not limited to, targeted agents, conventional chemotherapy agents, hormonal therapy agents, and supportive care agents. One or more therapeutic agents from the different classes, e.g., targeted, conventional chemotherapeutic, hormonal, and supportive care, and/or subclasses can be combined in the compositions described herein. The various classes described herein can be further divided into subclasses. By way of example, targeted agents can be separated into a number of different subclasses depending on their mechanism of action. As will be apparent to those of skill in the art, the agents can have more than one mechanism of action, and thus, could be classified into one or more subclasses. For purposes of the compositions and methods described herein, the following subclasses have been identified: anti-angiogenic, inhibitors of growth factor signaling, immunomodulators, inhibitors of protein synthesis, folding and/or degradation, inhibitors of gene expression, pro-apoptotic agents, agents that inhibit signal transduction and agents with “other” mechanisms of action. Typically, the mechanism of action for agents falling into the “other” subclass is unknown or poorly characterized.

For example, in some embodiments, targeted agents, such as bevacizumab, sutinib, sorafenib, 2-methoxyestradiol or 2ME2, finasunate, PTK787, vandetanib, aflibercept, volociximab, etaracizumab (MEDI-522), cilengitide, erlotinib, cetuximab, panitumumab, gefitinib, trastuzumab, TKI258, atacicept, alemtuzumab, aldesleukine, temsirolimus, everolimus, NPI-1387, MLNM3897, atiprimod, natalizumab, bortezomib, carfilzomib, NPI-0052, tanespimycin, saquinavir mesylate, ritonavir, nelfinavir mesylate, indinavir sulfate, belinostat, LBH589, AMG951, ABT-737, oblimersen, plitidepsin, SCIO-469, P276-00, enzastaurin, tipifamib, perifosine, imatinib, dasatinib, lenalidomide, thalidomide, simvastatin, and celecoxib can be administered prior to, concurrently with or after administration of NK cells and used to treat MM patients.

By way of another example, conventional chemotherapy agents, such as alklyating agents (e.g., oxaliplatin, carboplatin, cisplatin, cyclophosphamide, melphalan, ifosfamide, uramustine, chlorambucil, carmustine, mechloethamine, thiotepa, busulfan, temozolomide, dacarbazine), anti-metabolic agents (e.g., gemcitabine, cytosine arabinoside, Ara-C, capecitabine, 5FU (5-fluorouracil), azathioprine, mercaptopurine (6-MP), 6-thioguanine, aminopterin, pemetrexed, methotrexate), plant alkaloid and terpenoids (e.g., docetaxel, paclitaxel, vincristine, vinblastin, vinorelbine, vindesine, etoposide, VP-16, teniposide, irinotecan, topotecan), anti-tumor antibiotics (e.g., dactinomycin, doxorubicin, liposomal doxorubicin, daunorubicin, daunomycin, epirubicin, mitoxantrone, adriamycin, bleomycin, plicamycin, mitomycin C, caminomycin, esperamicins), and other agents (e.g., darinaparsin) can be administered prior to, concurrently with or after administration of NK cells and used to treat MM patients.

By way of another example, hormonal agents such as anastrozole, letrozole, goserelin, tamoxifen, dexamethasone, prednisone, and prednisilone can be administered prior to, concurrently with or after administration of NK cells and used to treat MM patients.

By way of another example, supportive care agents such as pamidronate, zoledonic acid, ibandronate, gallium nitrate, denosumab, darbepotin alpha, epoetin alpha, eltrombopag, and pegfilgrastim can be administered prior to, concurrently with or after administration of NK cells and used to treat MM patients.

The therapeutic agents can be administered in any manner found appropriate by a clinician and are typically provided in generally accepted efficacious dose ranges, such as those described in the Physician Desk Reference, 56th Ed. (2002), Publisher Medical Economics, New Jersey. In other embodiments, a standard dose escalation study can be performed to identify the maximum tolerated dose (MTD) (see, e.g., Richardson, et al. 2002, Blood, 100(9):3063-3067, the content of which is incorporated herein by reference).

In some embodiments, doses less than the generally accepted efficacious dose of a therapeutic agent can be used. For example, in various embodiments, the composition comprises a dosage that is less than about 10% to 75% of the generally accepted efficacious dose range. In some embodiments, at least about 10% or less of the generally accepted efficacious dose range is used, at least about 15% or less, at least about 25%, at least about 30% or less, at least about 40% or less, at least about 50% or less, at least about 60% or less, at least about 75% or less, and at least about 90%.

The therapeutic agents can be administered singly or sequentially, or in a cocktail with other therapeutic agents, as described below. The therapeutic agents can be administered orally, intravenously, systemically by injection intramuscularly, subcutaneously, intrathecally or intraperitoneally.

In some embodiments, the therapeutic agents are selected from the group consisting of dexamethasone, thalidomide, pomalidomide (Actimid™), vincristine, carmustine (BCNU), melphalan, cyclophosphamide, prednisone, doxorubicin, cisplatin, etoposide, bortezomib (Velcade®), lenalidomide (Revlimid®), ara-C, and/or combinations thereof.

In certain embodiments, the autologous NK cells are administered with a cytokine In some embodiments, the cytokine is selected from IL-2, IL-4, IL-7, IL-12 and IL-15.

Administration of one or more of the additional therapeutic agents described herein can be by any means known in the art, including, but not limited to, oral, rectal, nasal, topical (including buccal and sublingual) or parenteral (including subcutaneous, intramuscular, intravenous and intradermal) administration and will depend in part, on the available dosage form. For example, therapeutic agents that are available in a pill or capsule format typically are administered orally. However, oral administration generally requires administration of a higher dose than does intravenous administration. Determination of the optimal route of administration for a particular subject is well within the capabilities of those skilled in the art, and in part, will depend on the dose needed versus the number of times per month administration is required.

4.4 Effectiveness of Treatment Regimens

The use of expanded autologous NK cells as a monotherapy can be used to develop an effective treatment strategy based on the stage of myeloma being treated (see, e.g., Multiple Myeloma Research Foundation, Multiple Myeloma: Stem Cell Transplantation 1-30 (2004); U.S. Pat. Nos. 6,143,292, and 5,928,639, Igarashi et al. (2004) Blood 104(1): 170, Maloney et al. (2003) Blood 102(9):3447, Badros et al. (2002) J Clin Oncol. 20:1295, Tricot, et al. (1996), Blood 87(3):1196, the contents of which are incorporated herein by reference).

The staging system most widely used since 1975 is the Durie-Salmon system, in which the clinical stage of disease (Stage I, II, or III) is based on four measurements (see, e.g., Durie et al. (1975) Cancer, 36:842). These four measurements are: (1) levels of monoclonal (M) protein (also known as paraprotein) in the patient's serum and/or the urine; (2) the number of lytic bone lesions; (3) hemoglobin values; and, (4) serum calcium levels. The three stages can be further divided according to renal function, classified as A (relatively normal renal function, serum creatinine value <2.0 mg/dL) and B (abnormal renal function, creatinine value >2.0 mg/dL). A new, simpler alternative is the International Staging System (ISS) (see, e.g., Greipp et al., 2003, “Development of an international prognostic index (IPI) for myeloma: report of the international myeloma working group”, The Hematology). The ISS is based on the assessment of two blood test results, beta2-microglobulin and albumin, which categorizes patients into three prognostic groups irrespective of the type of therapy.

Treatment of multiple myeloma patients using the methods described herein typically elicits a beneficial response as defined by the European Group for Blood and Marrow transplantation (EBMT). Table 2 lists the EBMT criteria for response.

TABLE 2
EBMT/IBMTR/ABMTR¹ Criteria for Response

| | |
|---|---|
| Complete Response | No M-protein detected in serum or urine by |
| | immunofixation for a minimum of 6 weeks and |
| | fewer than 5% plasma cells in bone marrow |
| Partial Response | >50% reduction in serum M-protein level |
| | and/or 90% reduction in urine free light chain |
| | excretion or reduction to <200 mg/24 hrs for 6 |
| | weeks² |
| Minimal Response | 25-49% reduction in serum M-protein level |
| | and/or 50-89% reduction in urine free light |
| | chain excretion which still exceeds 200 mg/24 |
| | hrs for 6 weeks³ |
| No Change | Not meeting the criteria or either minimal |
| | response or progressive disease |
| Plateau | No evidence of continuing myeloma-related |
| | organ or tissue damage, <25% change in M- |
| | protein levels and light chain excretion for 3 |
| | months |
| Progressive Disease | Myeloma-related organ or tissue damage |
| | continuing despite therapy or its reappearance |
| | in plateau phase, >25% increase in serum M- |
| | protein level (>5 g/L) and/or >25% increase in |
| | urine M-protein level (>200 mg/24 hrs) and/ |
| | or >25% increase in bone marrow plasma cells (at |
| | least 10% in absolute terms)² |
| Relapse | Reappearance of disease in patients previously |
| | in complete response, including detection of |
| | paraprotein by immunofixation |

¹EBMT: European Group for Blood and Marrow transplantation; IBMTR: International Bone Marrow Transplant Registry; ABMTR: Autologous Blood and Marrow Transplant Registry.
²For patients with non-secretory myeloma only, reduction of plasma cells in the bone marrow by >50% of initial number (partial response) or 25-49% of initial number (minimal response) is required.
³In non-secretory myeloma, bone marrow plasma cells should increase by >25% and at least 10% in absolute terms; MRI examination may be helpful in selected patients.

Additional criteria that can be used to measure the outcome of a treatment include “near complete response” and “very good partial response”. A “near complete response” is defined as the criteria for a “complete response” (CR), but with a positive immunofixation test. A “very good partial response” is defined as a greater than 90% decrease in M protein (see, e.g., Multiple Myeloma Research Foundation, Multiple Myeloma Treatment Overview 9 (2005)).

The response of an individual clinically manifesting at least one symptom associated with multiple myeloma to the methods described herein depends in part, on the severity of disease, e.g., Stage I, II, or III, and in part, on whether the patient is newly diagnosed or has late stage refractory multiple myeloma. Thus, in some embodiments, treatment with autologous activated NK cells as a monotherapy elicits a complete response.

In other embodiments, treatment with autologous activated NK cells elicits a very good partial response or a partial response.

In various embodiments, treatment with autologous activated NK cells elicits a minimal response.

In other embodiments, treatment with autologous activated NK cells prevents the disease from progressing, resulting in a response classified as “no change” or “plateau” by the EBMT.

### 5. EXAMPLE 1

### Ex Vivo Expansion and Characterization of NK Cells from Multiple Myeloma Patients

5.1 Methods

Peripheral blood mononuclear cells (PMBC) from 8 patients with multiple myeloma were collected from blood samples by centrifugation on a Lymphoprep density step (Nycomed, Oslo, Norway), and were washed twice with unsupplemented RPMI medium and resuspended.

PMBC (1.5×10⁶) were incubated in a 24-well tissue culture plate for 14 days with 10⁶ irradiated K562 cells transfected with 4-1BBL ligand and membrane-bound IL-15 (K562-mb15-41BBL cells) in the presence of 300 U/ml of IL-2 in RPMI-1640 and 10% FCS. Medium was exchanged every 2 days with fresh medium and IL-2. After 7 days of co-culture, cells were restimulated by addition of 10⁶ irradiated modified K562 cells. The growth of NK cells, T cells and NKT cells in co-culture with K562-mb15-41BBL cells during the 14-day period was monitored by flow cytometry.

After expansion, cells were harvested and labeled with anti-CD3 fluorescein isothiocyanate (FITC) and anti-CD56 phycoerythrin (PE) antibodies. Non-expanded NK cells from the same patients were also labeled with the antibodies. Antibody staining of non-expanded and expanded NK cells was detected with a FACScan flow cytometer (Becton Dickinson). (See Imai et al. (2004) Leukemia 18:676; Ito et al. (1999) Blood 93:315; Srivannaboon et al. (2001) Blood 97:752).

NK cells were characterized by immunophenotyping using antibodies to the following molecules: NKp30, NKp44, NKp46, NK-p80, NKG2D and CD16 as described in Shi et al. (1008) Blood 111:1309.

5.2 Results

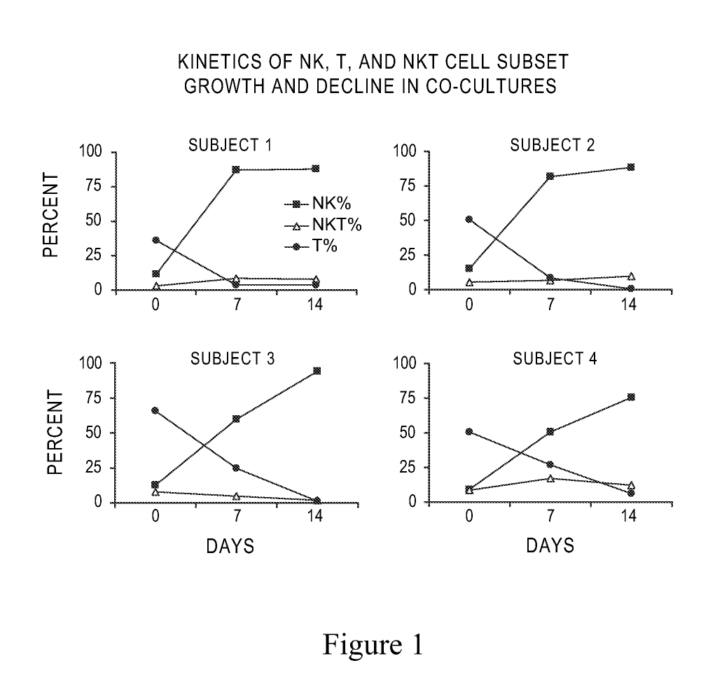

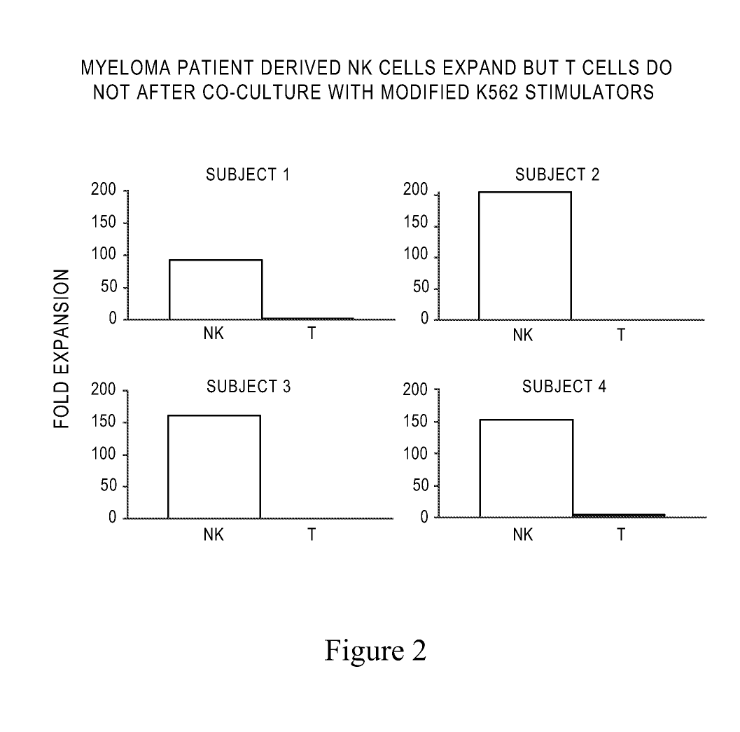

Over the 14-day culturing period, the number of NK cells expanded to account for over 75% of total cells in most of the ex vivo cultures, while the number of T-cells declined from around 25-50% to less than 10% of total cells. The number of NKT cells remained at a similarly low level (less than 10% of total cells) in all subjects. (FIG. 1) NK cells from four of the eight subjects showed significant expansion after 14 days of culturing (from 92-204-fold; average expansion 152-fold), while the number of T-cells in the ex vivo cultures did not expand. (FIG. 2)

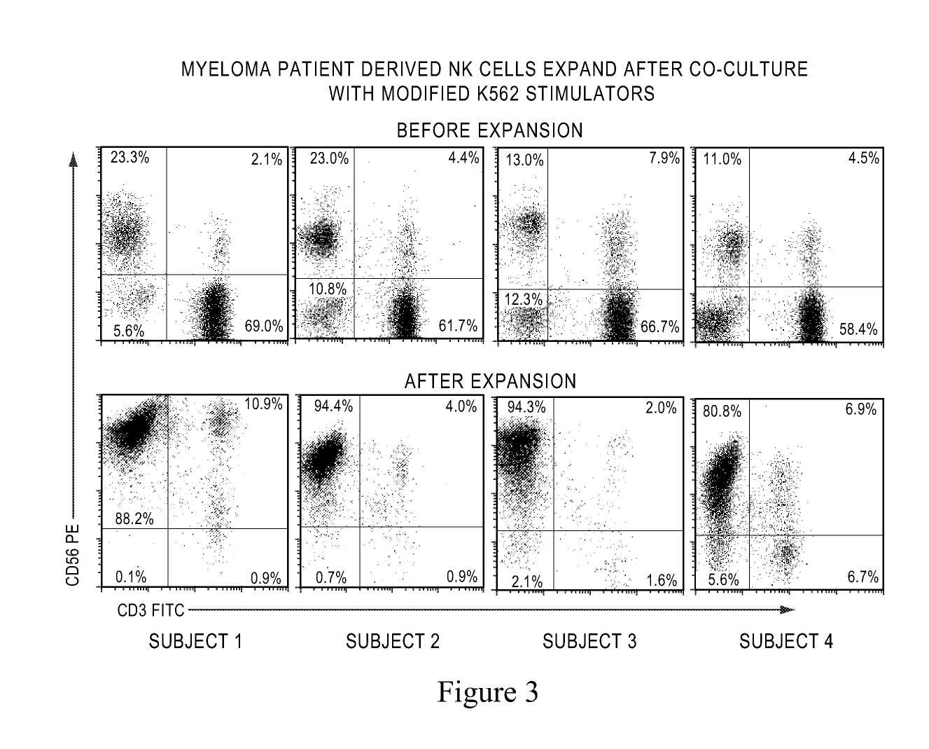

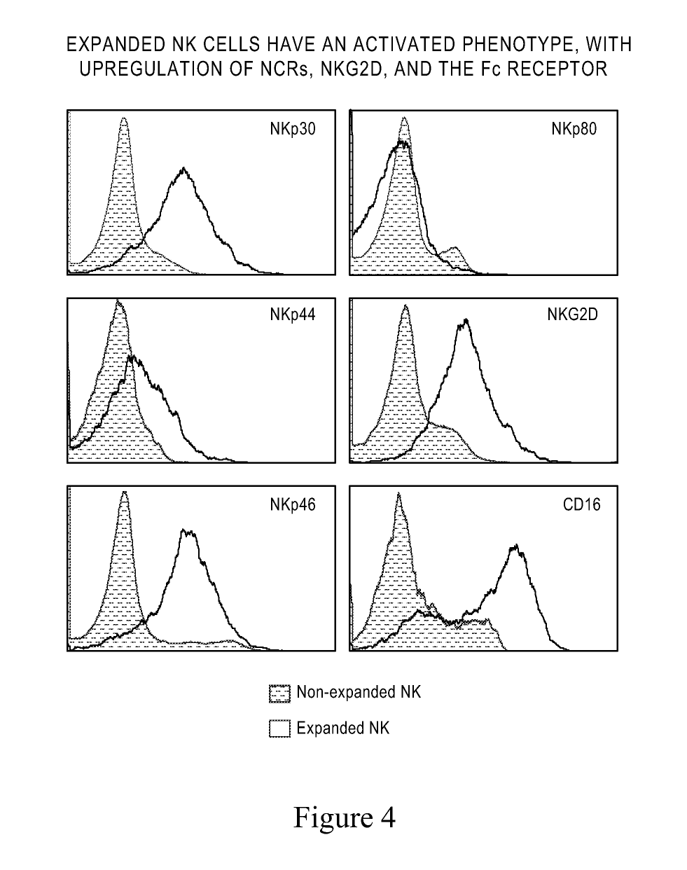

Non-expanded NK cells exhibited high expression of CD3 and low expression of CD65 on the cell surface. After expansion in the presence of modified K562 cells, NK cells showed high expression of CD65 and low expression of CD3. Expanded cells lacked T-cell receptors. (FIG. 3). Expanded NK cells from myeloma patients were found to express the NK-cell activating receptor NKG2D and natural cytotoxicity receptors NKp30, NKp44, and NKp46, indicating that the expanded NK cells are activated. (FIG. 4)

### 6. EXAMPLE 2

### Lysis of Multiple Myeloma Cells by Ex Vivo Expanded Autologous NK Cells

6.1 Methods

Target cells for this assay included (i) autologous PHA blasts; (ii) autologous CD34⁺ cells; (iii) autologous multiple myeloma cells; and (iv) K562 cells. Multiple myeloma cells from each subject were divided into the following treatment batches: (1) for treatment with expanded NK cells; and (2) for treatment with non-expanded NK cells. Target cells were cultured in vitro as previously described. See Colonna et al. (1993) Science 260:1121.

Target cells were labeled and the ⁵¹Cr release assay was performed as described in Colonna et al. (1993) Science 260:1121.

6.2 Results

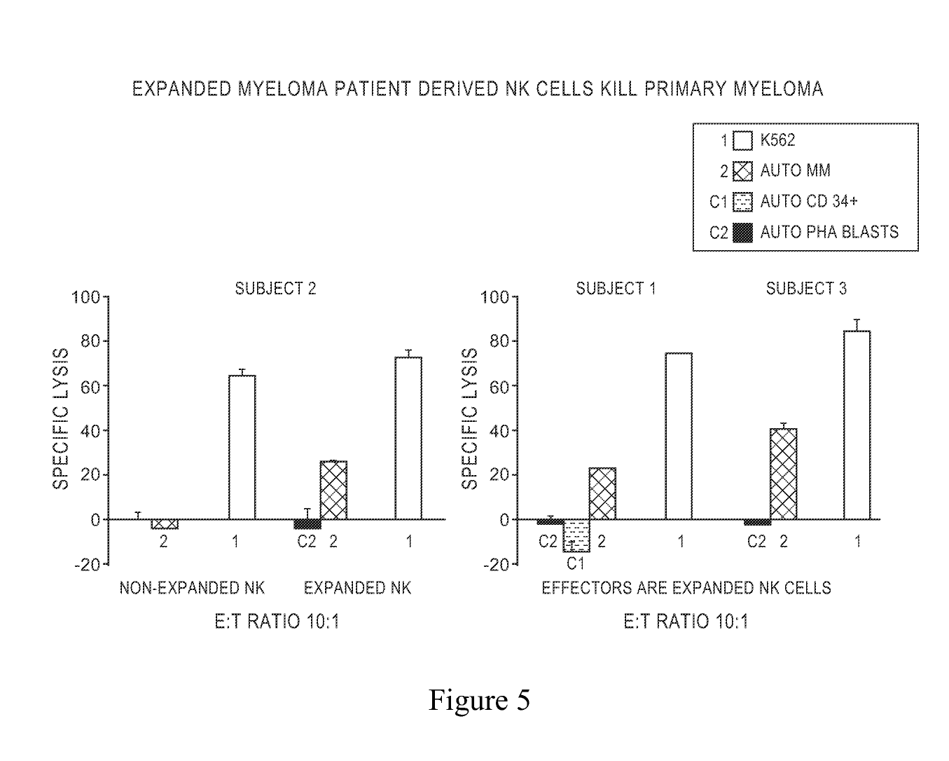

Expanded autologous NK cells killed on average about 30% of the total of cultured multiple myeloma cells from each of 3 subjects. The range of killing observed in the 3 subjects was 22-41% of cultured multiple myeloma cells. In contrast, no killing of multiple myeloma cells was observed with autologous NK cells that were not expanded or activated. (FIG. 5) Autologous PHA blasts and CD34+ stem cells were not killed.

### 7. EXAMPLE 3

### Distribution of Expanded NK Cells in the Bodies of Nod-Skid Mice

7.1 Methods

In order to determine the ability of ex vivo expanded NK cells to traffic to the bone marrow, activated NK cells were injected into the vein of NK cell depleted NOD-SCID mice, which were then sacrificed 0, 4 or 48 hours after injection. Peripheral blood, bone marrow and spleen tissue was harvested from each mouse and stained for flow cytometry. Samples were contacted with the following antibodies: anti-CD3 fluorescein isothiocyanate (FITC), anti-CD56 phycoerythrin (PE) and anti-CD45-PERCP. Antibody staining of peripheral blood, bone marrow and spleen tissue samples collected 0, 4 and 48 hours after injection was detected by flow cytometer.

7.2 Results

Activated NK cells (i.e., that express CD56, but not CD3) were detected in the bone marrow of mice at 48 hours after injection, indicating that NK cells traffic to the primary site of multiple myeloma in vivo.

### 8. EXAMPLE 4

### Treatment of Multiple Myeloma with Autologous NK Cells

A large volume leukapheresis to collect autologous PMBCs will be performed on patients prior to administration of the combination of expanded autologous NK cells and elotuzumab. PBMCs are co-cultured for one week in stem cell growth medium (CellGenix, Freiburg, Germany), or X-VIVO serum-free media (BioWhittaker, Verviers, Belgium), which can be supplemented with fetal bovine serum from certified sources or human serum from an AB blood donor, and to which an antibiotic such as gentamycin (50 mg/l) and from 10 to 1000 IU/ml human IL-2 are added. Irradiated K562-mb15-41BBL cells (30 Gy-100 Gy) are added at a ratio of 1:10 K562-mb15-41BBL:NK cells. Cells can be cultured in flasks or in bags (e.g., Teflon (FEP) bags, Baxter Lifecell bags or VueLife bags). Cells are fed after 2 and 5 days and harvested after 7 days of culture. The cell product is then depleted of residual T-cells using the CliniMACS System (Miltenyi), and cells are then washed and resuspended in PlasmaLyte-148 (Baxter, Deerfield, Ill.) with 0.5% human serum albumin. Expansion of CD56⁺CD3⁻ NK cells is about 90-fold.

Autologous NK cells will be transfused over approximately 8 hours by gravity. The target number of NK cells to be infused is 5×10⁵-4×10⁷ NK cells/kg. The recipient (i.e., subject) will receive standard monitoring for receiving cell products from a donor.

### 9. SPECIFIC EMBODIMENTS, CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. While various specific embodiments have been illustrated and described, it will be appreciated that various changes can be made without departing from the spirit and scope of the invention(s).

1. A method of treating multiple myeloma comprising administering to a human patient in need thereof an effective amount of expanded and activated autologous NK cells, wherein the autologous NK cells have been expanded and activated by culturing in the presence of K562 cells that express 4-1BBL and IL-15 on the cell surface, and wherein the expanded and activated NK cells are administered in the absence of an antibody that targets NK cells and of an antibody that targets myeloma cells.

2. The method of claim 1, further comprising before the administering step a step of culturing NK cells obtained from peripheral blood mononuclear cells of the patient in the presence of K562 cells that express 4-1BBL and IL-15 on the cell surface under conditions whereby the NK cells are expanded at least about 25-fold relative to the number of NK cells in the starting culture.

3. The method of claim 2, further comprising before the culturing step a step of isolating perphiperal blood mononuclear cells from the patient.

4. The method of claim 1, wherein the NK cells are cultured with from 10 to 1000 IU/ml human IL-2.

5. The method of claim 1, wherein the K562 cells are present in the autologous NK cell culture at a ratio of 1:10 K562 cells:NK cells.

6. The method of claim 1, wherein the autologous NK cells are expanded at least about 50-fold relative to the number of NK cells in the starting culture before expansion.

7. The method of claim 6, wherein the autologous NK cells are expanded at least about 100-fold relative to the number of NK cells in the starting culture before expansion.

8. The method of claim 7, wherein the autologous NK cells are expanded at least about 200-fold relative to the number of NK cells in the starting culture before expansion.

9. The method of claim 1, wherein the effective amount of autologous NK cells is from about 5×10⁵ mg/kg to about 5×10⁷ mg/kg of body weight of the subject.

10. The method of claim 1, wherein the autologous NK cells are administered intravenously.

11. The method of claim 1, wherein the autologous NK cells are administered with one or more additional agents.

12. The method of claim 11, wherein the autologous NK cells are administered with one or more additional agents.

13. The method of claim 12, wherein the one or more additional agents is a cytokine.

14. The method of claim 13, wherein the cytokine is selected from the group consisting of IL-2, IL-4, IL-7, IL-12 and IL-15.

15. The method of claim 12, wherein the one or more additional agents is a therapeutic agent.

16. The method of claim 15, wherein the therapeutic agent is selected from the group consisting of dexamethasone, thalidomide, pomalidomide (Actimid™), doxorubicin, bortezomib (Velcade®), lenalidomide (Revlimid®), and combinations thereof.

17. The method of claim 1, wherein the subject has undergone stem cell transplantation prior to the administration of the effective amount of autologous NK cells.

18. The method according to claim 17, wherein the stem cell transplantation is autologous stem cell transplantation.

19. The method according to claim 1, wherein said administration elicits a complete response as defined by the EBMT criteria for response.

20. The method according to claim 1, wherein said administration elicits a very good partial response as defined by the EBMT criteria for response.

21. The method according to claim 1, wherein said administration elicits a partial response as defined by the EBMT criteria for response.

22-27. (canceled)
